(19) United States
(12) Patent Application Publication
(10) Pub. No.: US 2025/0127978 A1
(43) Pub. Date: Apr. 24, 2025

(54) Semi-Disposable Medical-Fluid Waste Management Apparatus

(71) Applicant: Design Department, Inc., Franklin, WI (US)

(72) Inventors: Brian T. Leadingham, Pleasant Prairie, WI (US); Patrick C. Tetzlaff, Caledonia, WI (US)

(73) Assignee: Design Department, Inc., Franklin, WI (US)

(21) Appl. No.: 18/382,900

(22) Filed: Oct. 23, 2023

(51) Int. Cl. A61M 1/00

(52) U.S. Cl. CPC A61M 1/63; A61M 1/604; A61M 1/743

(57) ABSTRACT

A semi-disposable medical-fluid waste management apparatus includes a rigid canister having a first and second canister portions with an opening in the first portion. One of the canister portions includes an interstitial vacuum port therein, and the first and second canister portions include first and second seal portions, respectively, which when sealingly engaged form a canister joint in a canister-closed position. The canister joint defines a seal area, and the canister opening defines an opening area which is smaller than the seal area. The apparatus also includes (a) a disposable assembly having a lid configured to sealingly mate with the canister opening and lid-ports for vacuum and fluid connections and (b) a suction liner sealingly attached to the lid and to be inserted into the canister to receive medical-fluid waste when the canister is closed and the lid is mated with the canister opening.

### FIELD OF THE INVENTION

The present invention generally relates to the field of medical-fluid waste management in hospitals and more particularly to the suction-capture, handling, and disposal of such fluids in flexible containers.

### BACKGROUND OF THE INVENTION

In the hospital setting fluid management is a large and important activity. Nearly every operating room and most patient recovery spaces include provision for management and storage of waste fluids generated during surgical procedures and during patient recovery. The numbers associated with medical-fluid management are impressive. Recent estimates concluded that in 2022 there were just over 105 million surgical procedures performed in the United States alone, and that number is projected to grow substantially over the next few years.

Management, storage, and disposal of waste fluid, which may contain biohazards, need to be safe and effective but also need to be economical since the sheer size of the market results in large system-wide costs. From the viewpoint of economics, the size of the market translates into a need to address the efficiency and cost-effectiveness of each phase of the medical-fluid waste management lifecycle in order to ensure that costs can be managed efficiently. For example, disposal of medical-fluid containers, which store waste fluids, account for approximately 25% of all hospital plastic waste, and the production and disposal of all of this plastic translates into a significant impact on the environment as well. In addition, there is significant opportunity to reduce packaging costs, and shipping and inventory storage costs.

There are three primary categories of medical-fluid waste management products currently on the market. The first category is the rigid disposable single-use suction canisters which have been in the market for decades, distributed by healthcare companies such as Cardinal Health, Medline, DeRoyal, Bemis, and others. The second category is semi-disposable single-use systems including a reusable canister and disposable lid and canister liner for containment and subsequent removal of the waste fluid; these systems have also long been available, distributed by healthcare companies such as Cardinal Health, Serres, and Medline. These two categories of medical-fluid waste management products are usable for hospitals and medical facilities with sufficient facility vacuum systems. A third category, usable in facilities where facility vacuum is unavailable or insufficient, is all-in-one medical-fluid waste management systems which provide a vacuum source as well as fluid storage, systems such as the Neptune Waste Management System from Stryker Corporation.

Referring to the first category, the rigid disposable single-use suction canisters are generally produced by injection molding using commodity plastics such as low-density polyethylene (LDPE), crystal polystyrene, or polypropylene. Important parameters to consider are fluid storage capacity, connection to a vacuum source which creates a pressure differential between the canister and the surrounding environment. Not only does the container store fluid, it also functions as a pressure vessel, withstanding hospital vacuum, commonly regulated at 20 inches of mercury (˜10 psi). Rigid disposable suction canisters are generally considered safe and reasonably economical.

The third category, the all-in-one systems with their included vacuum source, while having the potential to offer a low-cost procedure, impose a substantial capital investment to purchase such systems and also tend to require significant training and maintenance to be successfully implemented.

It is the second category of medical-fluid waste management products, i.e., the semi-disposable single-use system, with its reusable rigid canister and disposable lid/liner assembly, to which the present invention pertains. Before describing the invention, further background information now follows.

Given that the rigid canister of this second category of systems is reusable, it can be made with more material, increasing its strength such that it can be subject to hospital vacuum over many years of usage. And, since the reusable rigid canister provides the structural-strength function of a pressure vessel, the disposable portion/assembly can be produced using less material, using the strength of the reusable canister to support a canister lid and a canister liner for containment and subsequent removal of the waste fluid. Such lids are typically injection-molded using a commodity plastic such as LDPE or polypropylene.

The liner portion of the disposable assembly is either blow-molded or produced using thin films (typically 4-6 mil thickness). The rigid canister is generally an open-top cylinder with a relatively flat bottom. The inner diameter of the canister is slightly larger than the disposable assembly such that the liner assembly can be dropped directly onto the canister. Since the lid has a larger diameter than the open top of the canister, the lid is prevented from passing through the opening into the canister.

Compared to the rigid disposable systems of the first category of medical-fluid waste management products, the semi-disposable systems use less material, and so generally cost less per medical procedure. However, downsides of semi-disposable systems are that current solutions do not optimize the cost of storage and transportation before use, and that current products also typically do not effectively provide a safe means of transporting the disposable assembly which contains waste fluid and has been removed from the rigid canister because the flexible nature of the liner makes it difficult to handle. And a tall, cylindrical flexible liner does not naturally rest in a stable position when removed from the canister and placed on a flat surface.

An additional issue with current semi-disposable systems is that their specific configurations create situations in which a significant amount of mechanical energy may be stored within the disposable assembly. Such mechanical energy is stored both by lid deflection caused by force resulting from the vacuum and by stretching or distention of the liner. If clinicians do not carefully follow the instructions for use, they can be exposed to a situation commonly referred to as canister reflux, in which the stored mechanical energy is rapidly released, spraying waste fluid over a wide area which may impact objects and personnel several feet around the disposable assembly.

A brief discussion of certain prior art now follows:

Serres Oy of Kauhajoki, Finland (www.serres.com) sells a series of disposable bags with lids and corresponding rigid suction canisters for medical-fluid waste management. Each suction bag is sealingly attached to a lid, forming the disposable assembly. In these systems, the configuration of the lids substantially matches that of the corresponding cylindrical canister such that when the bag is full and ready for disposal, the bag can be lifted back through the canister opening.

U.S. Pat. No. 3,556,101 (Economou) discloses a suction bottle assembly for medical-surgical use, including a generally rigid chamber, a disposable liner bag for insertion in the chamber, and a cover enclosing the chamber. U.S. Pat. No. 3,782,384 (Timmermans '384) also discloses a surgical suction jar having a rigid cylindrical chamber into which a flexible bag having vacuum and fluid connections is attached thereto. US Published Application 2009/0036847 (Rajamaki '847) discloses configurations but with specific fastening of the suction bag to the cover to keep the suction bag narrowed with respect to the suction canister. All three of these documents (Economou '101, Timmermans '384 and Rajamaki '847) disclose systems which, like the Serres Oy system, include canisters and liners/bags configured such that when the liner/bag is full and ready for disposal, it can be lifted back through the canister opening through which it was inserted.

A somewhat different configuration for a semi-disposable medical-fluid waste management system is disclosed in U.S. Pat. No. 11,285,255 (Korkeamaki et al. '255) which describes a medical-fluid waste management system having a suction bag attached to a handle with a largely linear seal, and the handle includes three inlet connections which pass through the linear seal rather than a cover as described in the previous documents.

None of these known systems includes the novel features of medical-fluid waste management systems disclosed in this application.

The inventive medical-fluid waste apparatus disclosed herein addresses these needs and opportunities within the market for semi-disposable medical-fluid waste management systems. The novel apparatus is configured to significantly reduce material content and volume of both disposable and non-disposable portions of the apparatus, to provide safe and convenient disposal of filled disposable liners, while maintaining compatibility with the existing systems which are presently installed in hospitals. In some embodiments of the novel apparatus, material content can be reduced by more than 50%, nearly 90% volume reduction of the disposable portion can be achieved, canister reflux is virtually eliminated, improving both clinician and patient safety, and the costs associated with packaging, transportation and storage of the semi-disposable assembly are dramatically reduced. In addition, the novel apparatus provides added simplicity of installation, setup, and liner disposal.

### OBJECTS OF THE INVENTION

It is an object of the novel medical-fluid waste management apparatus disclosed herein to provide apparatus which significantly reduces the amount of plastic material in both the disposable and non-disposable portions of the apparatus.

Another object of the medical-fluid waste management apparatus of this invention is to provide apparatus which reduces the weight and volume of both the disposable and non-disposable portions of the apparatus.

Another object of the present invention is to provide medical-fluid waste management apparatus which reduces the cost of packaging, transportation, and inventory storage of both the disposable and non-disposable portions of the apparatus.

Another object of the present invention is to provide medical-fluid waste management apparatus which essentially eliminates the risk of clinician and patient exposure to canister reflux by minimizing the amount of stored mechanical energy during usage.

Another object of the present invention is to provide medical-fluid waste management apparatus which eliminates the potential for liner distention.

Another object of the present invention is to provide medical-fluid waste management apparatus which substantially increases the safety and efficiency of disposal of the disposable portion of the apparatus.

Finally, yet another object is to provide medical-fluid waste management apparatus which meets the above objects while maintaining “standard” semi-disposable lid port number and sizes.

These and other objects will be apparent from the following descriptions and from the drawings.

### SUMMARY OF THE INVENTION

The invention disclosed herein is semi-disposable medical-fluid waste management apparatus with both non-disposable and disposable portions. In order to meet the objectives of the invention, both the non-disposable and disposable portions of the novel apparatus are structured to significantly reduce material content. The non-disposable portion comprises a two-piece rigid canister onto and into which the disposable portion, a canister lid with a suction liner bonded thereto, is assembled. The two portions of the rigid canister provide an airtight seal (canister seal) when assembled. The canister and lid are configured to permit an entire disposable portion, when filled with waste fluid, to be removed from the apparatus by being passed through the opening in the canister which receives the lid during usage. The disposable portion is then removed from the canister by opening the seal between the two portions of the canister. Spherically shaped canisters are particularly efficient in their use of material, both for volume and structural strength.

The inventive semi-disposable medical-fluid waste management apparatus includes a rigid canister having a first canister portion with a canister opening and a second canister portion. One of the first and second canister portions has an interstitial vacuum port therein, and the first and second canister portions include a first seal portion and a second seal portion, respectively, which when sealingly engaged form a canister joint in a canister-closed position. The canister joint defines a seal area, and the canister opening defines an opening area which is smaller than the seal area. The semi-disposable medical-fluid waste management apparatus also includes a disposable assembly which having a lid configured to sealingly mate with the canister at the canister opening and lid-ports for vacuum and fluid connections to the canister lid. The disposable assembly also includes a suction liner which is sealingly attached to the lid and configured to be inserted into the canister and to receive medical-fluid waste when the canister is in the canister-closed position and the lid is mated with the canister at the canister opening.

In preferred embodiments, the canister joint defines a joint plane, and the seal area is an area no less than any planar cross-section intersecting the canister parallel to the joint plane.

In some highly-preferred embodiments of the inventive apparatus, the lid is configured to pass into the first canister portion through the canister opening when not sealingly mated with the canister opening. In some highly-preferred embodiments, the disposable assembly is configured to be removed from the canister when the first and second canister portions are disengaged in a canister-open position.

In some highly-preferred embodiments of the inventive apparatus, the canister is substantially spherical, and some of these embodiments, the canister seal is circumferential.

In some highly-preferred embodiments of the apparatus, the suction liner is a suction bag formed by bonding together two sheets of flexible material, and in some of these embodiments, each of the two sheets is circular before the suction bag is formed.

In some embodiments, the canister of the apparatus includes a mounting connection, and in some embodiments, the first canister portion is a top canister portion, and the second canister portion is a bottom canister portion, and the mounting connection is on the bottom canister portion.

In some preferred embodiments, the lid of the apparatus includes pinch tabs configured to release the lid from the canister when the pinch tabs are depressed. In other preferred embodiments, the lid includes one or more tear-away tabs configured to release the lid from the canister when the one or more tear-away tabs are torn off the lid.

In some highly-preferred embodiments of the inventive apparatus, the canister includes a hinge between the first and second canister portions.

In some embodiments of the inventive semi-disposable medical-fluid waste management apparatus, the lid-ports include a fluid disposal port, a vacuum port, and a patient port, and some of these embodiments further include a tandem port for daisy-chaining the apparatus to other apparatus. Also, some of these embodiments include a porous vent plug in the vacuum port, configured to permit gas flow therethrough only when the plug is dry, and some embodiments include a one-way valve in the patient port configured to permit flow only into the suction liner.

Some embodiments of the apparatus include port caps configured to temporarily cover the lid-ports, and in some of these embodiments, the port caps are integrally formed as a port-cap assembly, and in some embodiments, the port-cap assembly includes the lid, and the port caps are tethered to the lid.

In another aspect of the inventive apparatus disclosed herein, the semi-disposable medical-fluid waste management apparatus includes a rigid canister having a convex outer wall formed as a body-of-revolution and defines a maximum cross-sectional area and a canister volume. The canister includes (a) an interstitial vacuum port and (b) a canister opening defining an opening area smaller than the maximum cross-sectional area. The apparatus includes a disposable assembly which has a lid configured to sealingly mate with the canister opening and includes lid-ports for vacuum and fluid connections to the canister lid. The disposable assembly of the apparatus also includes a suction bag which has a maximum empty-bag-volume, and the bag is sealingly attached to the lid and configured to be inserted into the canister and receive medical-fluid waste when the lid is mated with the canister opening. In some of these embodiments, the maximum empty-bag-volume is larger than the canister volume when the suction bag is filled within the canister.

In some embodiments of the inventive apparatus, the canister is spheroidal, and in some embodiments, the canister is spherical.

The term “semi-disposable” as used herein in describing the inventive apparatus means that a portion of the apparatus is disposable, the remaining portion being reused until the end of its useful life. In the apparatus disclosed herein, the canister lid and its attached suction liner are the main parts of a disposable assembly.

The term “seal” as used herein with respect to the inventive apparatus means that vacuum sealing is achieved at least to the degree defined (ISO) by Section 7.7.1 (Collection Containers for General Use) of the ISO 10079-3 standard for Medical Suction Equipment Powered From a Vacuum or Positive Pressure Gas Source.

The term “spheroidal” as used herein in describing a canister shape means an ellipsoid shape which is a sphere flattened at the poles such that cross-sections are elliptical defined by two radii. The longer radius is called the semi-major axis, and the shorter radius is called the semi-minor axis. When the two axes are equal, the spheroid is a sphere. This definition is a generally accepted mathematical definition for a spheroid.

The term “circumferential” as used herein with respect to a sphere indicates coincidence with the maximum cross-section of the sphere.

The term “interstitial vacuum port” as used here means a port which is configured to provide vacuum to the volume in a canister of the apparatus and outside of the suction liner, such volume being called interstitial volume.

### BRIEF DESCRIPTION OF THE DRAWINGS

FIGS. 1 through 6 are a set of perspective-view drawings illustrating a preferred embodiment of the semi-disposable medical-fluid waste management apparatus of this invention. FIG. 1 shows this preferred embodiment of the apparatus with the canister in the canister-closed position and the disposable assembly not yet inserted into the canister.

FIG. 2 illustrates the apparatus of FIG. 1 with the canister in the canister-closed position and the disposable assembly inserted into the canister.

FIG. 3 illustrates the apparatus of FIG. 2 with the vacuum port of the lid connected to a vacuum source.

FIG. 4 illustrates the apparatus of FIG. 2 with the canister in a partial canister-open position, the lid of the disposable assembly having passed through the canister opening into the canister.

FIG. 5 illustrates the apparatus of FIG. 4 with the canister in the canister-open position.

FIG. 6 illustrates the apparatus of FIG. 5 with the disposable assembly removed from the canister.

FIGS. 7A through 7E are a set of two-dimensional drawings illustrating various views of the apparatus of FIG. 3. FIG. 7A is a side elevation view of the apparatus of FIG. 3.

FIG. 7B is a top elevation view of the apparatus of FIG. 3.

FIG. 7C is a cross-sectional view of the disposable assembly of FIG. 3 taken at section C-C in FIG. 7B.

FIG. 7D is a cross-sectional view of the disposable assembly of FIG. 3 taken at section D-D in FIG. 7C.

FIG. 7E is an enlarged detail drawing of DETAIL 1 in FIG. 7C.

FIG. 8A is a top-view elevation drawing of the disposable assembly of the semi-disposable medical-fluid waste management apparatus of this invention.

FIG. 8B is a cross-sectional view of the disposable assembly of FIG. 8A, taken at section B-B in FIG. 8A.

FIG. 8C is a cross-sectional view of the disposable assembly of FIG. 8A, taken at section C-C in FIG. 8A.

FIG. 9 is an exploded perspective-view drawing of the disposable assembly of FIG. 1.

FIG. 10 is a perspective-view drawing of an alternative embodiment for tethering port caps to a canister lid.

FIGS. 11A through 11F are a set of drawings illustrating an alternative embodiment of a canister lid which has tear-away tabs integrally formed with the lid. FIG. 11A is a perspective view drawing of a tear-away lid assembly.

FIG. 11B is a cross-sectional view of the tear-away lid assembly of FIG. 11A shown in place on a canister opening.

FIG. 11C is an enlarged detail drawing of DETAIL 2 of FIG. 7C.

FIG. 11D is another perspective view drawing of the tear-away tab assembly of FIG. 11A.

FIG. 11E is a perspective view drawing of a tear-away ring after the tear-away tabs have been torn from the tear-away assembly of FIG. 11D.

FIG. 11F is a perspective view drawing of a tear-away lid after the tear-away tabs have been torn from the tear-away assembly of FIG. 11D.

FIGS. 12 through 14 illustrate three alternative embodiments for the inventive apparatus. FIG. 12 is a perspective-view drawing of a first alternative embodiment of the semi-disposable medical-fluid waste management apparatus of this invention in which the canister has a convex outer wall formed as a body-of-revolution and the disposable assembly not yet inserted into the canister. The alternative embodiments in FIGS. 12-14 are shown with a pull-away lid in the disposable assembly of the apparatus.

FIG. 13 is a perspective-view drawing of a second alternative embodiment of the semi-disposable medical-fluid waste management apparatus of this invention in which the canister is spheroidal.

FIG. 14 is a perspective-view drawing of a third alternative embodiment of the semi-disposable medical-fluid waste management apparatus of this invention in which the canister is spherical.

### DETAILED DESCRIPTION OF PREFERRED EMBODIMENTS

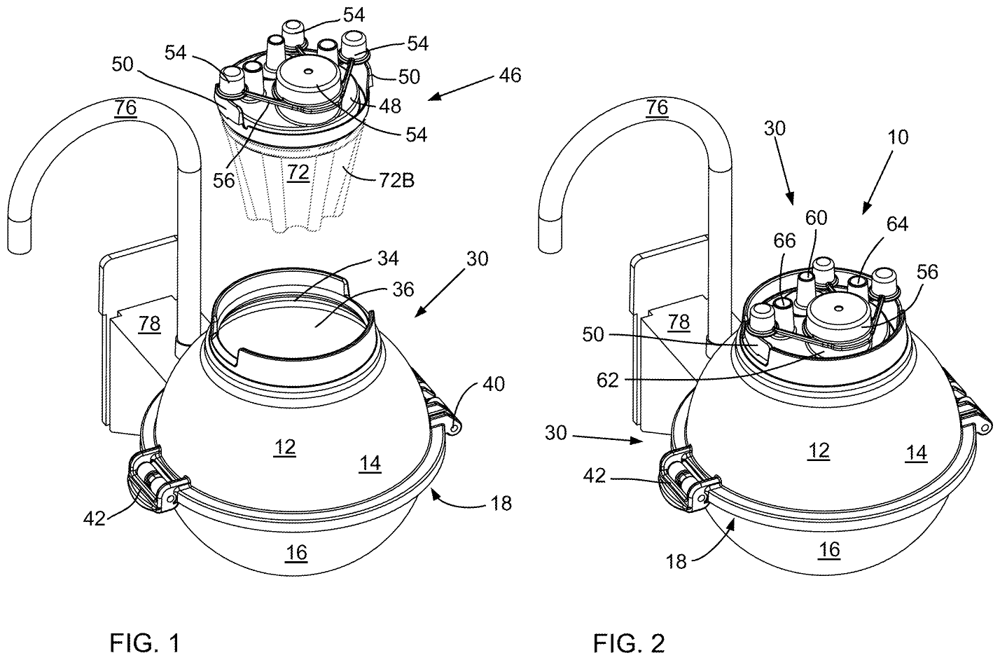

FIGS. 1 through 9 illustrate one embodiment of the inventive semi-disposable medical-fluid waste management apparatus disclosed herein. FIGS. 10A through 13 illustrate alternative embodiments of various elements of the inventive apparatus. In all of the figures in this application, reference numbers are not shown in every instance of each component in order to reduce the complexity of the drawings.

FIGS. 1 through 6 are six perspective-view drawings illustrating a preferred embodiment 10 of the semi-disposable medical-fluid waste management apparatus 10 of this invention. (For simplicity, reference number 10 is used to indicate both the apparatus and its embodiment.) Apparatus 10 includes a rigid canister 12 having a first canister portion 14 with a canister opening 34 which defines an opening area 36. First canister portion 14 also includes a first seal portion 22. Canister 12 also includes a second canister portion 16 having an interstitial vacuum port 38 and a second seal portion 24 which is configured to sealingly engage first seal portion 22, forming a canister joint 18 when canister 12 is in a canister-closed position 30 as shown at least in FIGS. 1-3. Canister joint 18 defines a seal area 26, opening area 36 being smaller than seal area 26.

Canister joint 18 includes a canister hinge 40 connecting first and second canister portions 14 and 16 and a canister joint fastener 42, together configured to permit canister 12 to open and close like a clam shell and be locked in canister-closed position 30.

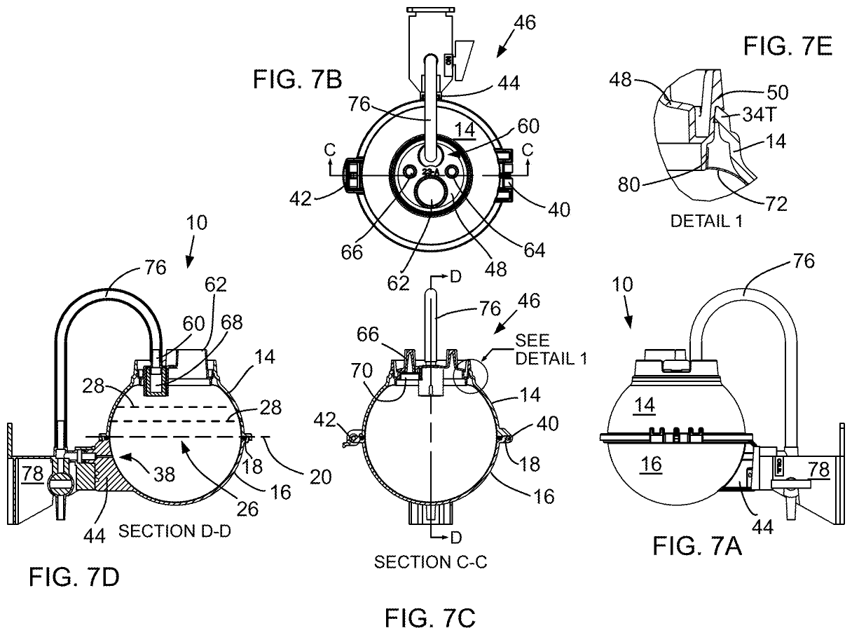

Apparatus 10 also includes a disposable assembly 46 which includes a lid 48 configured to sealingly mate with canister opening 34. Lid 48 also has lid-ports for vacuum and fluid connections to canister 12 during operation. In apparatus 10, such lid-ports include a vacuum lid-port 60, a fluid disposal lid-port 62, a patient lid-port 66, and a tandem lid-port 64. Disposable assembly 46 also includes a suction liner 72 (in embodiment 10, suction liner 72 is a suction bag 72B) which is sealingly attached to lid 48. Suction bag 72B is shown in FIG. 1 in a packed or gathered state so that suction bag 72B can be inserted into canister 12 through canister opening 34 while canister 12 is in canister-closed position 30 and lid 48 can be sealingly mated to canister 12 at canister opening 34 as shown in FIG. 2. Lid 48 includes two opposingly-mounted pinch tabs 50 which are configured to prevent lid 48 from passing through canister opening 34, but when displaced inwardly with a pinching action, permit lid 48 to pass into canister 12 through canister opening 34. Details of the configuration of pinch tabs 50 are illustrated in FIGS. 7C and 7E.

FIGS. 1-6 also illustrate disposable assembly 46 having a port-cap assembly 56 which includes four port caps 54, one for each of lid-ports 60, 62, 64, and 66, used to temporarily cover a respective lid-port. For convenience, port-cap assembly 56 may be an integrally-formed component, as shown in FIGS. 1-6 and FIG. 9, and port-cap assembly 56 may also be tethered to lid 48 by one or more port-cap tethers 58 as shown in FIGS. 1-3, 5-6, and 9. FIGS. 1 and 2 illustrate port-caps 54 off vacuum lid-port 60 patient lid-port 66, and tandem lid-port 64, and on fluid disposal lid-port 62. FIG. 10 illustrates an alternative embodiment 56A (port-cap/lid assembly 56A) for tethering port caps 54 in apparatus 10 in which port caps 54 are tethered directly to lid 48 via tethers 58.

In FIGS. 1-6, apparatus 10 is shown being mounted on a mounting connection receiver 78 via a mounting connection 44 on second canister portion 16. (See FIGS. 7A and 7D for the relationship among these components not fully shown in FIGS. 1-6.) Mounting connection receiver 78 may be mounted on a wall (not shown) or a movable pole (also not shown) as required for the medical procedure being performed.

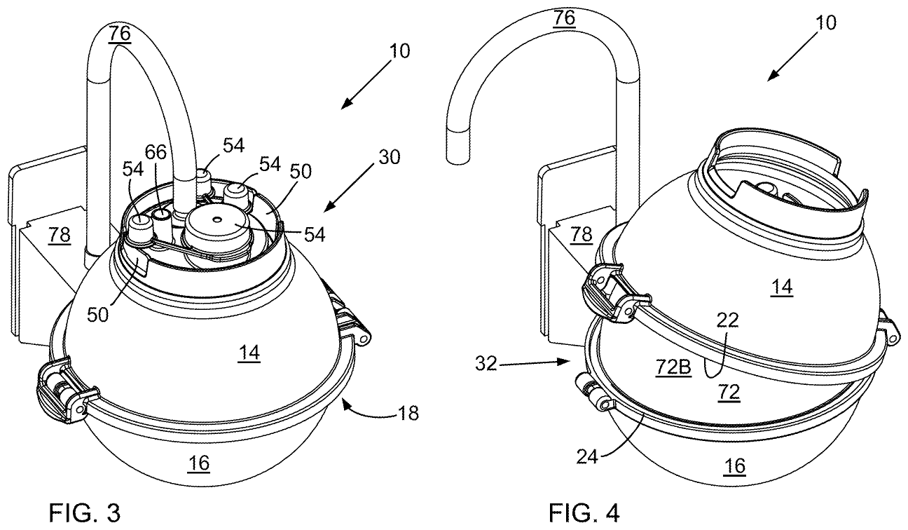

FIG. 3 illustrates a vacuum source connector 76 (vacuum source not shown) connected to vacuum lid-port 60 after canister 12 has been placed in canister-closed position 30 as shown in FIG. 2. FIG. 3 also shows vacuum lid-port 60 and patient lid-port 66 each not covered with a respective port cap 54 and fluid-disposal lid-port 62 and tandem lid-port 64 each covered by a respective port cap 54. As such, FIG. 3 illustrates apparatus 10 ready to receive a vacuum connection (not shown) and a patient connection (also not shown), placing apparatus 10 ready to receive medical-fluid waste into suction bag 72B during a medical procedure. Note that interstitial vacuum port 38 is connected to the same vacuum source (not shown) that provides vacuum to apparatus 10 via vacuum source connector 76. Connection to interstitial vacuum port 38 is illustrated in FIGS. 7A and 7D.

After the medical procedure is completed, vacuum source connector 76 is disconnected, along with the connection (not shown) to patient lid-port 66, and port caps 54 may be placed on vacuum lid-port 60 and patient lid-port 66. Then, an operator pinches tabs 50 as described above, permitting lid 48 to pass through canister opening 34 and into canister 12. Then, canister 12 is opened via releasing canister joint fastener 42 and lifting first canister portion 14 from second canister portion 16 into a partial canister-open position 32. As illustrated in FIG. 4, suction bag 72B may be full of waste fluid, as shown, or may be partially full of waste fluid.

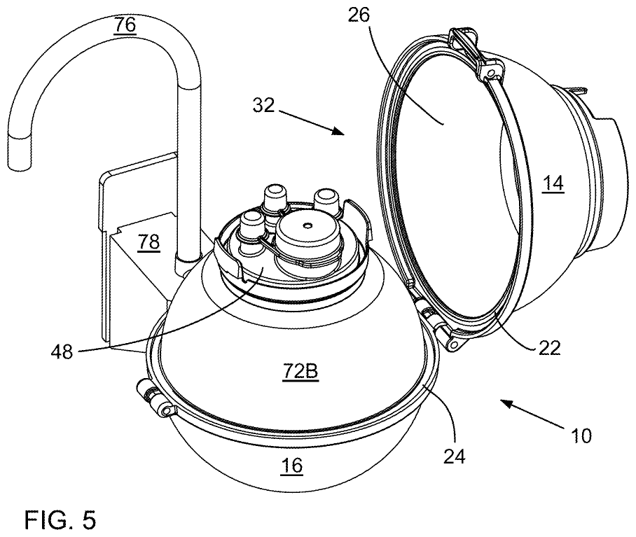

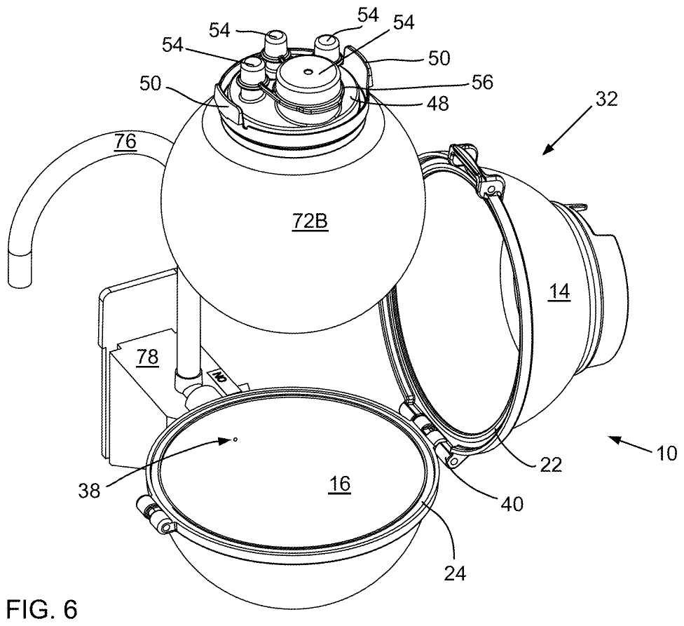

FIG. 5 illustrates canister 12 in the canister-open position 32 and suction bag 72B full of waste fluid, ready to be lifted from second canister portion 16 for appropriate disposal, as illustrated in FIG. 6.

Apparatus 10 may include a one-way valve 70 aligned in patient lid-port 66 to permit waste fluid to flow only into suction bag 72B. One-way valve 70 in patient lid-port 66 is shown in FIGS. 7C, 8B, 9 and 11B. Apparatus 10 may also include a porous plug 68 in vacuum lid-port 60 which permits the flow of gases through lid-port 60 only if porous plug 68 remains dry. If porous plug 68 becomes wet, gas flow is prevented in either direction through vacuum lid-port 60. Porous plug 68 in vacuum lid-port 60 is shown in FIGS. 7D, 8C, and 9.

Referring now to FIGS. 7A through 7E, FIG. 7A primarily illustrates the mounting of apparatus 10 to mounting connection receiver 78 through mounting connection 44 which is part of second canister portion 16. Mounting connection receiver 78 may be configured for mounting on a variety of objects as needed, such as a wall (not shown but as configured in the drawings), accessory pole (not shown), and other convenient location.

FIG. 7B is a top elevation view of apparatus 10, and FIG. 7C is a cross-section view C-C as indicated in FIG. 7B. In turn, FIG. 7C includes a section line D-D which indicates the sectional view D-D as shown in FIG. 7D. FIG. 7D illustrates a joint plane 20 defined by canister joint 18. As described above, canister joint 18 also defines seal area 26 which is, therefore, the area in joint plane 20 within canister joint 18. FIG. 7D also illustrates planar exemplary cross-sections 28 (two shown) parallel to joint plane 20. In embodiment 10, the area of any such planar cross-section 28 of canister 12 is smaller than seal area 26.

FIG. 7C also illustrates in more detail the configuration of sealingly mating lid 48 with canister opening 34, indicating the location of DETAIL 1 shown in FIG. 7E. As described above, lid 48 includes two pinch tabs 50, and a portion of one pinch tab 50 is shown in FIG. 7E engaging a portion of a canister-opening top lip 34T which prevents lid 48 from passing into canister 12, also aligning lid 48 for sealingly mating with canister opening 34. As can be seen in FIG. 7E, when pinch tabs 50 are pinched toward one another, canister-opening top lip 34T is cleared to permit lid 48 to pass through canister opening 34 and into canister 12.

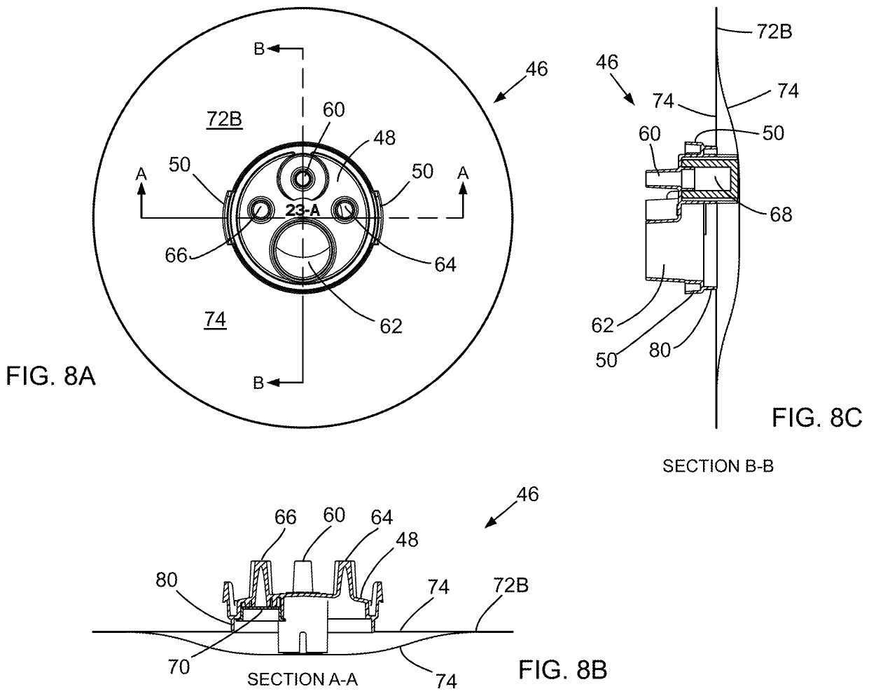

FIGS. 8A through 8C are two-dimensional views of disposable assembly 46. FIG. 8A is a top-view elevation drawing, and FIGS. 8B and 8C are cross-sectional views of disposable assembly 46 taken at section lines B-B and C-C, respectively. FIG. 8A illustrates that suction bag 72B includes circular sheets 74, which are preferably made of polymer material. The cross-sectional views of FIGS. 8B and 8C illustrate that suction bag 72B includes two sheets 74 which form suction bag 72B, and that suction bag 72B is bonded to lid 48 by a liner/lid sealing attachment 80.

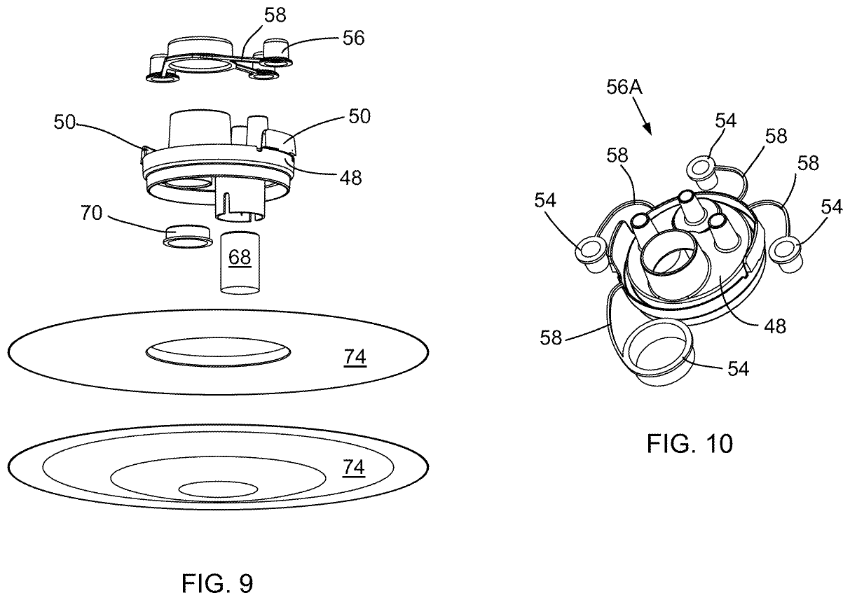

FIG. 9 is an exploded perspective-view drawing of disposable assembly 46 along with port-cap assembly 56. (Note that the three inner circles within the circular sheet 74 at the bottom of FIG. 9 are indicating that such sheet 74 is shown as non-planar due to the structure of lid 48 as shown in FIGS. 8B and 8C.)

FIG. 10 is a perspective-view drawing of an alternative port-cap/lid assembly 56A for tethering port caps 54 to canister lid 48. Port-cap/lid assembly 56A includes four tethers 58, one for each of the four port caps 54, and is integrally formed into a single assembly 56A.

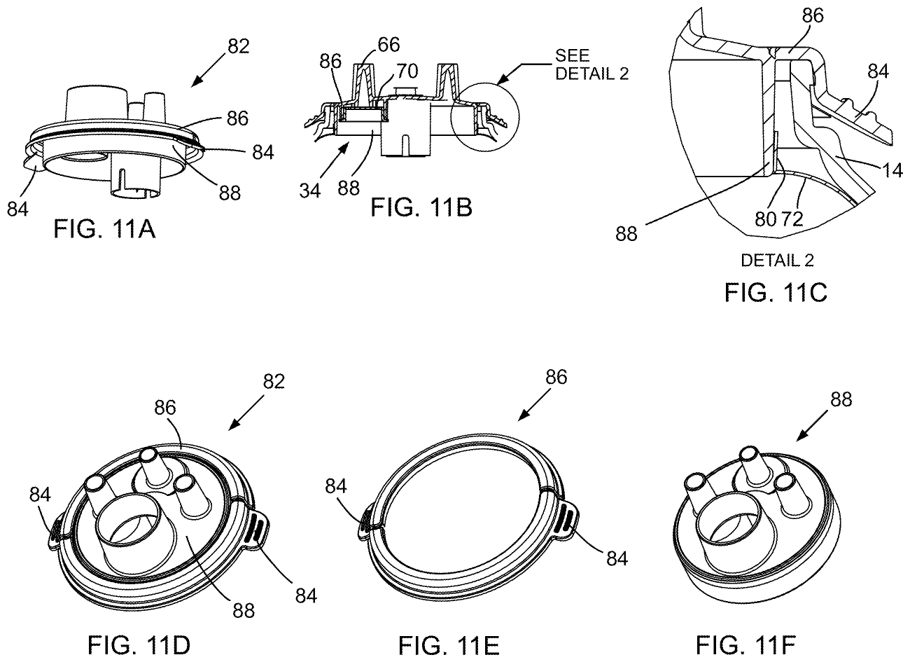

FIGS. 11A through 11F are six drawings illustrating an embodiment 82 of a tear-away lid assembly (also indicated by reference number 82) which includes two tear-away tabs 84 as a tear-away tab ring 86 integrally formed with tear-away lid 88. FIG. 11A is a perspective view drawing of tear-away lid assembly 82, and FIG. 11B is a cross-sectional view of tear-away lid assembly 82 shown in place on canister opening 34. FIG. 11C is an enlarged detail drawing of DETAIL 2 as indicated in FIG. 7B. As seen best in FIG. 11C, tear-away tab ring 86 positions tear-away lid 88 on first canister portion 14 and prevents tear-away lid 88 from passing through canister opening 34 into canister 12. When tear-away tabs 84 are torn away from tear-away lid 88, tear-away lid 88 is able to be passed through canister opening 34 into canister 12 for subsequent disposal.

FIGS. 11D through 11F provide additional perspective views of tear-away lid assembly 82, tear-away tab ring 86, and tear-away lid 88, respectively.

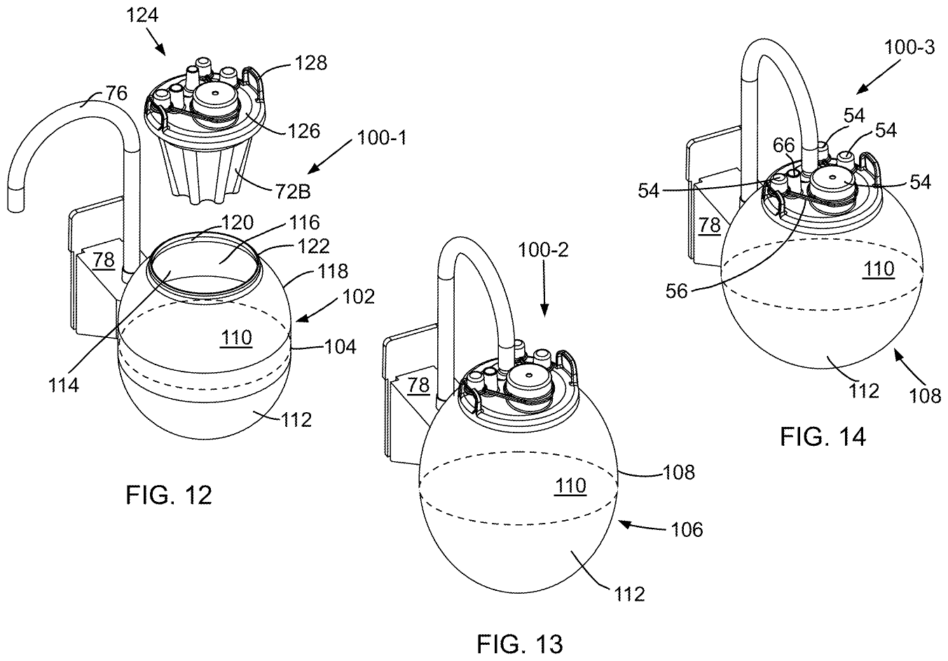

FIGS. 12 through 14 illustrate three alternative embodiments of the inventive semi-disposable medical-fluid waste management apparatus. FIG. 12 illustrates a first alternative embodiment 100-1 having a rigid canister 102 having a convex outer wall 104 formed as a body-of-revolution canister (also 102) and defining a maximum cross-sectional area 110 and a canister volume 112. (Note that maximum cross-sectional areas 110 and canister volumes 112 are indicated in each of FIGS. 12-14 but that this does not imply that the areas and volumes, respectively, are identical in each of such figures but that each embodiment has such properties.)

FIG. 13 illustrates a second alternative embodiment 100-2 having a rigid canister 106 formed as a spheroidal canister (also 106) which defines maximum cross-sectional area 110 and canister volume 112. Similarly, FIG. 14 illustrates a third alternative embodiment 100-3 having a rigid canister 108 formed as a spherical canister (also 108) which also defines maximum cross-sectional area 110 and canister volume 112. (Note that in each of FIGS. 12-14, a dotted line is used to illustrate maximum cross-sectional area 110.)

Canisters 102, 106, and 108, in FIGS. 12, 13 and 14, respectively, each include an interstitial vacuum port (not shown but the same as interstitial vacuum port 38 as shown in FIG. 6 in apparatus 10), and each such canister also includes a canister opening 114 defining an opening area 116 which is smaller than maximum cross-sectional area 110 in each of embodiments 100-1, 100-2, and 100-3.

Embodiments 100-1, 100-2, and 100-3 each also include a disposable assembly 124 which includes a pull-away lid 126 which has a vacuum lid-port 60, a fluid disposal port 62, a patient port 66, and a tandem port 64, and each such port is provided with a port cap 54, port caps 54 being integrally formed into a port-cap assembly 56. Disposable assembly 124 includes a suction bag 72B as shown in FIG. 12 in a pre-use configuration prior to insertion of disposable assembly 124 into canister 102. Suction bag 72B has a maximum empty-bag-volume (not shown), and suction bag 72B is sealingly attached to pull-away lid 126 having pulls 128 and configured to be inserted into each of canisters 102, 106, and 108 and to receive medical-fluid waste when pull-away lid 126 is mated with its respective canister 102, 106, and 108. The maximum empty-bag-volume is larger than canister volume 112 when suction bag 72B is filled within its respective canister.

FIG. 12 also illustrates that body-of-revolution canister 102 has a canister body 118, a lid-mating surface 122, and an outwardly-projecting portion 120 between lid-mating surface 122 and canister body 118. Outwardly-projecting portion 120 enables a full or nearly-full suction bag 72B to pass more easily through canister opening 114.

While the principles of this invention have been described in connection with specific embodiments, it should be understood clearly that these descriptions are made only by way of example and are not intended to limit the scope of the invention.

1. Semi-disposable medical-fluid waste management apparatus, the apparatus comprising:
a rigid canister having a first canister portion with a canister opening and a second canister portion, one of the first and second canister portions having an interstitial vacuum port therein, the first and second canister portions including a first seal portion and a second seal portion, respectively, which when sealingly engaged form a canister joint in a canister-closed position, the canister joint defining a seal area and the canister opening defining an opening area smaller than the seal area; and
a disposable assembly comprising:
a lid configured to sealingly mate with the first canister portion at the canister opening and having lid-ports for vacuum and fluid connections thereto; and
a suction liner sealingly attached to the lid and configured to be inserted into the canister and receive medical-fluid waste when the canister is in the canister-closed position and the lid is mated with the canister.

2. The semi-disposable medical-fluid waste management apparatus of claim 1 wherein the canister joint defines a joint plane.

3. The semi-disposable medical-fluid waste management apparatus of claim 2 wherein the seal area is an area no less than any planar cross-section intersecting the canister parallel to the joint plane.

4. The semi-disposable medical-fluid waste management apparatus of claim 1 wherein the lid is configured to pass into the first canister portion through the canister opening when not sealingly mated with the canister.

5. The semi-disposable medical-fluid waste management apparatus of claim 1 wherein the disposable assembly is configured to be removed from the canister when the first and second canister portions are disengaged in a canister-open position.

6. The semi-disposable medical-fluid waste management apparatus of claim 1 wherein the canister is substantially spherical.

7. The semi-disposable medical-fluid waste management apparatus of claim 6 wherein the canister seal is circumferential.

8. The semi-disposable medical-fluid waste management apparatus of claim 1 wherein the suction liner is a suction bag formed by bonding together two sheets of flexible material.

9. The semi-disposable medical-fluid waste management apparatus of claim 8 wherein each of the two sheets is circular before the suction bag is formed.

10. The semi-disposable medical-fluid waste management apparatus of claim 1 wherein the canister includes a mounting connection.

11. The semi-disposable medical-fluid waste management apparatus of claim 1 wherein the first canister portion is a top canister portion, and the second canister portion is a bottom canister portion.

12. The semi-disposable medical-fluid waste management apparatus of claim 11 wherein the canister includes a mounting connection.

13. The semi-disposable medical-fluid waste management apparatus of claim 12 wherein the mounting connection is on the bottom canister portion.

14. The semi-disposable medical-fluid waste management apparatus of claim 1 wherein the lid further includes pinch tabs configured to release the lid from the canister when the pinch tabs are depressed.

15. The semi-disposable medical-fluid waste management apparatus of claim 1 wherein the lid further includes one or more tear-away tabs configured to release the lid from the canister when the one or more tear-away tabs are torn off the lid.

16. The semi-disposable medical-fluid waste management apparatus of claim 1 wherein the canister includes a hinge between the first and second canister portions.

17. The semi-disposable medical-fluid waste management apparatus of claim 1 wherein the lid-ports include a fluid disposal port, a vacuum port, and a patient port.

18. The semi-disposable medical-fluid waste management apparatus of claim 17 wherein the lid-ports further include a tandem port for daisy-chaining the apparatus to other apparatus.

19. The semi-disposable medical-fluid waste management apparatus of claim 17 further including a porous vent plug in the vacuum port, configured to permit gas flow therethrough only when the plug is dry.

20. The semi-disposable medical-fluid waste management apparatus of claim 17 further including a one-way valve in the patient port configured to permit flow only into the suction liner.

21. The semi-disposable medical-fluid waste management apparatus of claim 17 wherein the disposable assembly further includes port caps configured to temporarily cover the lid-ports.

22. The semi-disposable medical-fluid waste management apparatus of claim 21 wherein the port caps are integrally formed as a port-cap assembly.

23. The semi-disposable medical-fluid waste management apparatus of claim 22 wherein the port-cap assembly includes the lid, and the port caps are tethered to the lid.

24. Semi-disposable medical-fluid waste management apparatus, the apparatus comprising:
a rigid canister having a convex outer wall formed as a body-of-revolution and defining a maximum cross-sectional area and a canister volume, the canister including (a) an interstitial vacuum port and (b) a canister opening defining an opening area smaller than the maximum cross-sectional area; and
a disposable assembly comprising:
a lid configured to sealingly mate with the canister at the canister opening and including lid-ports for vacuum and fluid connections thereto; and
a suction bag sealingly attached to the lid and configured to be inserted into the canister and receive medical-fluid waste when the lid is mated with the canister.

25. The semi-disposable medical-fluid waste management apparatus of claim 24 wherein the canister is spheroidal.

26. The semi-disposable medical-fluid waste management apparatus of claim 25 wherein the canister is spherical.

27. The semi-disposable medical-fluid waste management apparatus of claim 24 wherein the suction bag is formed by bonding together two sheets of flexible material.

28. The semi-disposable medical-fluid waste management apparatus of claim 25 wherein each of the two sheets is circular before the suction bag is formed.

29. The semi-disposable medical-fluid waste management apparatus of claim 24 wherein the canister has a lid-mating surface and an outwardly-projecting portion between the lid-mating surface and the canister.

30. The semi-disposable medical-fluid waste management apparatus of claim 24 wherein the lid-ports include a fluid disposal port, a vacuum port, and a patient port.

31. The semi-disposable medical-fluid waste management apparatus of claim 30 further including a tandem port for daisy-chaining the apparatus with other apparatus.

32. The semi-disposable medical-fluid waste management apparatus of claim 30 further including a porous vent plug in the vacuum port, configured to permit gas flow therethrough only when the plug is dry.

33. The semi-disposable medical-fluid waste management apparatus of claim 30 further including a one-way valve in the patient port configured to permit flow only into the canister.

34. The semi-disposable medical-fluid waste management apparatus of claim 24 wherein the lid includes port caps for temporary covering of each port.

35. The semi-disposable medical-fluid waste management apparatus of claim 34 wherein the port caps are formed as a port-cap assembly.

36. The semi-disposable medical-fluid waste management apparatus of claim 35 wherein the port-cap assembly includes the lid, and the port caps are tethered to the lid.

37. The semi-disposable medical-fluid waste management apparatus of claim 24 wherein the suction bag has a maximum empty-bag-volume which is larger than the canister volume when the suction bag is filled within the canister.
